(19) United States
(12) Patent Application Publication
(10) Pub. No.: US 2012/0083459 A1
(43) Pub. Date: Apr. 5, 2012

(54) USE OF GANGLIOSIDE TO DECREASE PROPAGATION OF MALIGNANT PROSTATE CELLS

(75) Inventors: Michael Thomas CLANDININ, Edmonton (CA); John MIKLAVCIC, Edmonton (CA); Vera MAZURAK, Edmonton (CA)

(73) Assignee: THE GOVERNORS OF THE UNIVERSITY OF ALBERTA, Edmonton (CA)

(21) Appl. No.: 13/250,167

(22) Filed: Sep. 30, 2011

Related U.S. Application Data
(60) Provisional application No. 61/388,701, filed on Oct. 1, 2010.

(51) Int. Cl. A61K 31/7028; A61P 35/00; C07H 15/10

(52) U.S. Cl. 514/25; 536/17.9

(57) ABSTRACT

Uses of exogenous ganglioside to inhibit the propagation of prostate cancer cells are provided. Gangliosides regulate many cellular processes including cell death. The present disclosure assesses the role of ganglioside in prostate cell growth. Malignant prostate (PC-3) and control (RWPE-1) cells can be cultured with or without ganglioside treatment. Cells can be assayed for differences in cell growth. Supplementation with ganglioside (GD3) can decrease growth of PC-3 cells by 30% compared to controls (p<0.01). Ganglioside can have therapeutic benefit in prostate cancer as demonstrated by decreased growth of malignant PC-3 cells.

### CROSS-REFERENCE TO RELATED APPLICATION

This application claims the benefit of U.S. Provisional Application No. 61/388,701, filed on Oct. 1, 2010, and said earlier application is incorporated herein by reference in its entirety for continuity of disclosure.

### TECHNICAL FIELD

This disclosure relates to methods of inhibiting the propagation of prostate cancer cells.

### BACKGROUND

Prostate cancer (“CaP”) is the most prevalent cancer in North American men. The diagnosis of CaP evokes patient anxiety and subsequently, 90% of men elect for immediate treatment. About half of treated cases elect for radical prostatectomy. This treatment requires a patient to accept diminished quality of life thereafter and does not guarantee freedom from recurrence.

Watchful waiting and active surveillance are CaP management strategies that monitor tumor characteristics over time and prompt intervention at signs of increased growth. This period of surveillance provides opportunity for nutritional intervention. Due to the long latency period of CaP, it would be beneficial to determine if bioactive components in foods can reduce cancer cell growth. Many dietary supplements analyzed to date (ex. lycopene, vitamin E) have not scientifically demonstrated significant decreases in CaP cell growth.

Accordingly, there is a need to provide methods of inhibiting the propagation of prostate cancer cells that overcome the shortcomings of the prior art.

### BRIEF SUMMARY

Uses of exogenous ganglioside to inhibit the propagation of prostate cancer cells are provided. Gangliosides regulate many cellular processes including cell death. The present disclosure assesses the role of ganglioside in prostate cell growth. Malignant prostate (“PC-3”) and control (“RWPE-1”) cells can be cultured with or without ganglioside treatment. Cells can be assayed for differences in cell growth. Supplementation with ganglioside (“GD3”) can decrease growth of PC-3 cells by 30% compared to controls (p<0.01). Ganglioside can have therapeutic benefit in prostate cancer as demonstrated by decreased growth of malignant PC-3 cells.

Incorporated by reference in its entirety into this application is a paper written by the within inventors/applicants entitled, “Effects of Ganglioside on Growth and Cell Surface Ganglioside Densities in Prostate Cancer In Vitro”, published as Chapter 3 of the thesis entitled “Ganglioside Increases Metastatic Potential and Susceptibility of Prostate Cancer to Gene Therapy In Vitro” by John Miklavcic, University of Alberta, Department of Agricultural, Food and Nutritional Science; Edmonton, Alberta, Canada, Fall 2009.

Broadly stated, in some embodiments, a use of exogenous ganglioside is provided to inhibit the propagation of prostate cancer cells.

Broadly stated, in some embodiments, a dietary supplement is provided comprising exogenous ganglioside wherein the dietary supplement is used to inhibit the propagation of prostate cancer cells.

Broadly stated, in some embodiments, a composition is provided comprising exogenous ganglioside wherein the composition is used to inhibit the propagation of prostate cancer cells.

Broadly stated, in some embodiments, a method of treatment to inhibit the propagation of prostate cancer cells is provided, the method comprising the administration of exogenous ganglioside to an individual in need of such treatment.

Broadly stated, in some embodiments, a method of inhibiting the propagation of prostate cancer cells is provided, the method comprising contacting the prostate cancer cells with exogenous ganglioside.

Broadly stated, in some embodiments, the use of exogenous ganglioside is provided in the manufacture of a medicament to be used to inhibit the propagation of prostate cancer cells.

Broadly stated, in some embodiments, a kit is provided to inhibit the propagation of prostate cancer cells, the kit comprising exogenous ganglioside and instructions for use of the exogenous ganglioside.

In some embodiments, methods and compositions of exogenous ganglioside to inhibiting the propagation of prostate cancer cells are provided wherein the exogenous ganglioside comprises GD3-enriched zeta dairy lipid powder which is administered to a subject as a dietary supplement in a dosage of at least 30 mg/mL, the inhibition of the propagation of prostate cancer cells is accomplished by apoptosis, the prostate cancer cells comprising PC-3 cells, the propagation of normal prostate cells is not inhibited, and the normal prostate cells comprising RWPE-1 cells.

### BRIEF DESCRIPTION OF THE DRAWING

FIG. 1 is a bar graph depicting the decrease of growth of PC-3 cells in vitro, as compared to control cell lines as a function of dosage of a ganglioside treatment.

### DETAILED DESCRIPTION

Uses and compositions of exogenous ganglioside to inhibit the propagation of prostate cancer cells are disclosed herein.

In some embodiments, exogenous ganglioside can be used to inhibit the propagation of prostate cancer cells. In some embodiments, exogenous ganglioside can be provided to an individual as a dietary supplement. The dietary supplement can be provided in any appropriate form, mode, or method as would be understood by one skilled in the art. In some embodiments, exogenous ganglioside can be provided to an individual diagnosed with prostate cancer to control tumor growth. In some embodiments, exogenous ganglioside can be provided to an individual not diagnosed with prostate cancer to control propagation of prostate cancer cells as a preventative measure.

In some embodiments, exogenous ganglioside can be provided to an individual as a daily dietary supplement. In some embodiments, exogenous ganglioside can be provided to an individual at a concentration slightly higher than physiologically relevant. As one skilled in the art would appreciate, the concentration of exogenous ganglioside provided can be varied appropriately to inhibit the propagation of prostate cancer cells.

In some embodiments, the exogenous ganglioside can comprise GD3. In some embodiments, the exogenous ganglioside can comprise GD3-enriched zeta dairy lipid powder. In some embodiments, the exogenous ganglioside can be taken up by prostate cancer cells as would be understood by those skilled in the art. In some embodiments, the exogenous ganglioside can induce apoptosis as would be understood by those skilled in the art. In some embodiments, apoptosis can inhibit the propagation of prostate cancer cells. In some embodiments, the inhibition of the propagation of prostate cancer cells can prevent or reduce prostate tumor growth.

In some embodiments, the prostate cancer cells inhibited can comprise PC-3 cells. As would be understood by one skilled in the art, PC-3 cells can serve as classical models for human prostate cancer.

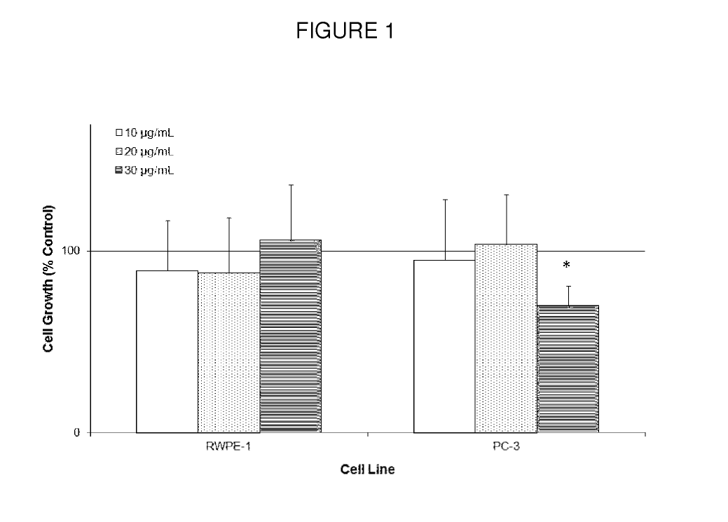

Referring now to FIG. 1, in some embodiments, exogenous ganglioside can be used to inhibit the propagation of prostate cancer cells while the propagation of normal prostate cells is not inhibited. In some embodiments, the normal prostate cells can comprise RWPE-1 cells.

In some embodiments, a dietary supplement for use to inhibit the propagation of prostate cancer cells can comprise exogenous ganglioside. In some embodiments, a composition comprising exogenous ganglioside can be used for use to inhibit the propagation of prostate cancer cells.

In one embodiment, the use of exogenous ganglioside to inhibit the propagation of prostate cancer cells could be presented as a kit comprising a dietary supplement and instructions for use of the dietary supplement.

The following examples and FIGURE are provided to aid the understanding of the present disclosure, the true scope of which is set forth in the claims. It is understood that modifications can be made in the procedures set forth without departing from the spirit or scope of the invention.

### Example 1

### Growth Conditions

RWPE-1 and PC-3 cell lines were obtained from the American Type Culture Collection. Cell line characteristics are outlined in Table 1. Cultures were maintained in Costar™ 3516 six-well tissue culture treated plates. RWPE-1 cells were cultured in Keratinocyte-SFM containing L-glutamine, supplemented with bovine pituitary extract (193 μL/100 mL) and human recombinant epidermal growth factor (0.591 μL/100 mL). Medium was replaced two-three times/week and cells were subcultivated (1:6) every seven days. PC-3 cells were cultured in Ham's F-12 Nutrient Mixture containing L-glutamine and supplemented with NaHCO₃ (1.18 g/L). Medium was replaced three times/week and cells were subcultivated (1:5) every 7 days. To passage cells, monolayers were rinsed once with phosphate-buffered saline (PBS) before being dislodged by cell lifter into fresh medium. Cells were grown in standard culture incubation conditions of 37° C. and 5% atmospheric CO₂. All cell cultures were supplemented with 1% (v/v) pooled human serum and 1% (v/v) antibiotic/antimycotic (penicillin, streptomycin, amphotericin B).

TABLE 1
Cell lines employed in current study

| | | Androgen | PSA | |
|---|---|---|---|---|
| Cell Line | Type | Responsiveness | Producing | Origin |
| RWPE-1 | Normal | Yes | Yes | Normal Human |
| | | | | Prostate Cells |
| PC 3 | Tumour | No | No | Human Bone Metastasis |

### Example 2

### Ganglioside Extraction

Ganglioside was extracted from GD3-enriched zeta dairy lipid powder (Fonterra, Cambridge NZ) by modified Folch method (12, 13). Powder (0.50 g) was added to 30 mL of chloroform/methanol (C/M; 2:1 v/v), vortexed (30 sec), and shaken (>2 hr). 0.025% (w/v) CaCl₂/double-distilled (dd) H₂O was added to samples before inversing several times. Samples were spun (1,000 rpm for 10 min at room temperature) and the upper layer was withdrawn and filtered through a Sep-Pak Classic C18 cartridge. Cartridges were rinsed with 10 mL ddH₂O before eluting ganglioside with 2 mL of methanol, followed by 10 mL of C/M (2:1 v/v). C/M was removed under N₂ gas before ganglioside was redissolved in 500 μL C/M (2:1 v/v) and stored at 4° C. before quantifying. Ganglioside extract is composed of 4% GM3, 92% GD3, and 4% unknown (Table 2).

TABLE 2
Ganglioside composition of zeta dairy lipid powder

| Ganglioside | Relative % |
|---|---|
| GM3 | 3.86 |
| GD3 | 92.2 |
| Unknown | 3.92 |

### Example 3

### Quantification

Samples were redissolved in C/M (2:1 v/v) after drying under N₂ gas. Aliquots (10 μL) were taken in duplicate and dried under N₂ gas before adding 500 μL ddH₂O and vortexing. Resorcinol-HCl (500 μL) was added to test tubes; then capped, vortexed, and heated (8 min at 160° C.). After cooling to room temperature, 500 μL of butylacetate/butanol (85:15 v/v) was added to test tubes and vortexed. The upper layer was withdrawn and read by a spectrophotometer (8452A, Hewlett Packard) at 580 nm. Ganglioside quantitification was determined by relating absorbance values to an authentic N-acetyl neuraminic acid standard curve. Samples were dried under nitrogen gas and suspended in appropriate cell culture medium to the desired concentration before filter sterilization (0.22 μm).

### Example 4

### Cell Growth Assay

Cells were grown to 60% confluence before replacing medium with fresh medium containing 0, 10, 20, or 30 μg/mL of ganglioside. After 48 hr, cells were rinsed once with PBS and harvested with 0.25% trypsin-EDTA for 5-10 min before inactivation with FBS. Cell counts were estimated by trypan blue exclusion using a haemacytometer. High viability (>95%) was obtained for each experiment. Cell counts in ganglioside-supplemented groups were computed as a percentage relative to cell counts in the non-supplemented group.

There was no difference in cell viability between treatment and control groups. Ganglioside did not alter cell growth in RWPE-1 cells at concentrations of 10, 20, or 30 μg/mL. At 30 μg/mL, a 30% reduction (p<0.01) in the number of PC-3 cells was observed (FIG. 1).

Ganglioside can decrease growth of PC-3 cells in vitro. Treatment group counts were calculated as a percentage of control group counts (n≧5). The asterisk in FIG. 1 indicates significant (p<0.05) difference from 100%. Results of FIG. 1 are summarized in Table 3.

TABLE 3
Summary of cell growth data

| Dose (μg/mL) | RWPE-1 | p-value | PC-3 | p-value |
|---|---|---|---|---|
| 10 | 89.28 | NS | 95.07 | NS |
| 20 | 88.00 | NS | 103.6 | NS |
| 30 | 105.9 | NS | 69.84 | <0.01 |

p-values denote significant difference from 100%, NS=not significant.

### Example 5

### Statistics

Observations were made in duplicate or triplicate across microtitre plate wells. Each observation was obtained from a consecutive cell passage. A t-test for proportion means was conducted to determine whether ganglioside treatment altered cell growth relative to untreated control. Means for cell surface ganglioside absorbance were computed for treatment and control groups and compared using Student's paired t-test.

Although a few embodiments have been shown and described, it will be appreciated by those skilled in the art that various changes and modifications might be made without departing from the spirit or scope of the invention. Unless defined otherwise, all technical and scientific terms used herein have the same meaning as commonly understood to one of ordinary skill and the art to which this invention belongs. In addition, the terms and expressions used in this specification have been used herein as terms of description and not of limitation, and there is no intention in the use of such terms and expressions of excluding equivalents of the features shown and described or portions thereof, it being recognized that the scope of the invention is defined and limited only by the appended claims.

### REFERENCES

The following documents are hereby incorporated by reference into this application in their entirety.
1. Leskawa K C, Erwin R E, Leon A, Toffano G, Hogan E L. Incorporation of exogenous ganglioside GM1 into neuroblastoma membranes: Inhibition by calcium ion and dependence upon membrane protein. Neurochem Res 1989; 14(6):547-54.
2. Tomasi M, Roda L G, Ausiello C, et al. Interaction of GMI ganglioside with bovine serum albumin: Formation and isolation of multiple complexes. Eur J Biochem 1980; 111(2):315-24.
3. Tognon G, Frapolli R, Zaffaroni M, et al. Fetal bovine serum, but not human serum, inhibits the in vitro cytotoxicity of ET-743 (yondelis, trabectedin), an example of potential problems for extrapolation of active drug concentrations from in vitro studies. Cancer Chemother Pharmacol 2004; 53(1):89-90.
4. Facci L, Leon A, Toffano G, Sonnino S, Ghidoni R, Tettamanti G. Promotion of neuritogenesis in mouse neuroblastoma cells by exogenous gangliosides. relationship between the effect and the cell association of ganglioside GM1. J Neurochem 1984; 42(2):299-305.
5. Vihko P, Herrala A, Harkonen P, et al. Control of cell proliferation by steroids:The role of 17HSDs. Mol Cell Endocrinol 2006; 248(1-2):141-8.
6. Soronen P, Laiti M, Torn S, et al. Sex steroid hormone metabolism and prostate cancer. J Steroid Biochem Mol Biol 2004; 92(4):281-6.
7. Libertini S J, Tepper C G, Rodriguez V, Asmuth D M, Kung H J, Mudryj M. Evidence for calpain-mediated androgen receptor cleavage as a mechanism for androgen independence. Cancer Res 2007; 67(19):9001-5.
8. McVary K T, McKenna K E, Lee C. Prostate innervation. Prostate Suppl 1998; 8:2-13.
9. Pratt V C, Watanabe S, Bruera E, et al. Plasma and neutrophil fatty acid composition in advanced cancer patients and response to fish oil supplementation. Br J Cancer 2002; 87(12):1370-8.
10. Hurdle HL. Examination of in vitro prostate cancer models supplemented with lycopene, vitamine E and fish oil. 2006.
11. Malisan F, Testi R. GD3 ganglioside and apoptosis. Biochim Biophys Acta 2002; 1585(2-3):179-87.
12. Schnabl K L, Larcelet M, Thomson A B, Clandinin M T. Uptake and fate of ganglioside GD3 in human intestinal CaCo-2 cells. Am J Physiol Gastrointest Liver Physiol 2009.
13. Folch J, Lees M, Sloane M, Stanley G H. A simple method for the isolation and purification of total lipides from animal tissues. J Biol Chem 1957; 226(1):497-509.
14. Ravindranath M H, Bauer P M, Cornillez-Ty C, Garcia J, Morton D L. Quantitation of the density of cell surface carbohydrate antigens on cancer cells with a sensitive cell-suspension ELISA. J Immunol Methods 1996; 197(1-2):51-67.
15. Ma R, Koulov A, Moulton C, et al. Apoptosis of human breast carcinoma cells in the presence of disialosyl gangliosides: II. treatment of SKBR3 cells with GD3 and GD1b gangliosides. Glycoconj J 2004; 20(5):319-30.
16. Tempera I, Buchetti B, Lococo E, et al. GD3 nuclear localization after apoptosis induction in HUT-78 cells. Biochem Biophys Res Commun 2008; 368(3):495-500.
17. Omran O M, Saqr H E, Yates A J. Molecular mechanisms of GD3-induced apoptosis in U-1242 MG glioma cells. Neurochem Res 2006; 31(10):1171-80.
18. Alessandri G, Filippeschi S, Sinibaldi P, et al. Influence of gangliosides on primary and metastatic neoplastic growth in human and murine cells. Cancer Res 1987; 47(16):4243-7.
19. Malisan F, Franchi L, Tomassini B, et al. Acetylation suppresses the proapoptotic activity of GD3 ganglioside. J Exp Med 2002; 196(12):1535-41.
20. Kniep B, Kniep E, Ozkucur N, et al. 9-O-acetyl GD3 protects tumor cells from apoptosis. Int J Cancer 2006; 119(1):67-73.
21. Furukawa K, Thampoe I J, Yamaguchi H, Lloyd K O. The addition of exogenous gangliosides to cultured human cells results in the cell type-specific expression of novel surface antigens by a biosynthetic process. J Immunol 1989; 142(3):848-54.
22. Popa I, Pons A, Mariller C, et al. Purification and structural characterization of de-N-acetylated form of GD3 ganglioside present in human melanoma tumors. Glycobiology 2007; 17(4):367-73.
23. Park E J, Suh M, Clandinin M T. Dietary ganglioside and long-chain polyunsaturated fatty acids increase ganglioside GD3 content and alter the phospholipid profile in neonatal rat retina. Invest Ophthalmol Vis Sci 2005; 46(7):2571-5.
24. Prinetti A, Basso L, Appierto V, et al. Altered sphingolipid metabolism in N-(4-hydroxyphenyl)-retinamide-resistant A2780 human ovarian carcinoma cells. J Biol Chem 2003; 278(8):5574-83.
25. Sorensen L K. A liquid chromatography/tandem mass spectrometric approach for the determination of gangliosides GD3 and GM3 in bovine milk and infant formulae. Rapid Commun Mass Spectrom 2006; 20(24):3625-33.
26. Cheresh D A, Pytela R, Pierschbacher M D, Klier F G, Ruoslahti E, Reisfeld R A. An arg-gly-asp-directed receptor on the surface of human melanoma cells exists in an divalent cation-dependent functional complex with the disialoganglioside GD2. J Cell Biol 1987; 105(3):1163-73.
27. Ohkawa Y, Miyazaki S, Miyata M, Hamamura K, Furukawa K, Furukawa K. Essential roles of integrin-mediated signaling for the enhancement of malignant properties of melanomas based on the expression of GD3. Biochem Biophys Res Commun 2008; 373(1):14-9.
28. Sun J, Shaper N L, Itonori S, Heffer-Lauc M, Sheikh K A, Schnaar R L. Myelin-associated glycoprotein (siglec-4) expression is progressively and selectively decreased in the brains of mice lacking complex gangliosides. Glycobiology 2004; 14(9):851-7.
29. Ladisch S, Kitada S, Hays E F. Gangliosides shed by tumor cells enhance tumor formation in mice. J Clin Invest 1987; 79(6):1879-82.
30. Valentino L A, Ladisch S. Localization of shed human tumor gangliosides: Association with serum lipoproteins. Cancer Res 1992; 52(4):810-4.
31. Li R X, Ladisch S. Shedding of human neuroblastoma gangliosides. Biochim Biophys Acta 1991; 1083(1):57-64.
32. Radsak K, Schwarzmann G, Wiegandt H. Studies on the cell association of exogenously added sialo-glycolipids. Hoppe Seylers Z Physiol Chem 1982; 363(3):263-72.
33. Ravindranath M H, Muthugounder S, Presser N, Ye X, Brosman S, Morton D L. Endogenous immune response to gangliosides in patients with confined prostate cancer. Int J Cancer 2005; 116(3):368-77.
34. Ravindranath M H, Tsuchida T, Morton D L, Irie R F. Ganglioside GM3:GD3 ratio as an index for the management of melanoma. Cancer 1991; 67(12):3029-35.
35. Hyuga S, Yamagata S, Tai T, Yamagata T. Inhibition of highly metastatic FBJ-LL cell migration by ganglioside GD1a highly expressed in poorly metastatic FBJ-S1 cells. Biochem Biophys Res Commun 1997; 231(2):340-3.
36. Hyuga S, Yamagata S, Takatsu Y, et al. Suppression by ganglioside GD1A of migration capability, adhesion to vitronectin and metastatic potential of highly metastatic FBJ-LL cells. Int J Cancer 1999; 83(5):685-91.

We claim:

1. A composition comprising exogenous ganglioside wherein the composition is used to inhibit the propagation of prostate cancer cells.

2. The composition of claim 1 wherein the exogenous ganglioside comprises GD3.

3. The composition of claim 2 wherein the GD3 comprises GD3-enriched zeta dairy lipid powder.

4. The composition of claim 1 wherein the prostate cancer cells comprise PC-3 cells.

5. The composition of claim 1 wherein propagation of normal prostate cells is not inhibited.

6. The composition of claim 1 wherein the composition comprises exogenous ganglioside in a dosage of at least 30 μg/mL.

7. A method of treatment to inhibit the propagation of prostate cancer cells, the method comprising the administration of exogenous ganglioside to an individual in need of such treatment.

8. The method of claim 7 wherein the exogenous ganglioside is administered to the individual as a dietary supplement.

9. The method of claim 7 wherein the exogenous ganglioside comprises GD3.

10. The method of claim 9 wherein the GD3 comprises GD3-enriched zeta dairy lipid powder.

11. The method of claim 7 wherein the prostate cancer cells comprise PC-3 cells.

12. The method of claim 7 wherein propagation of normal prostate cells is not inhibited.

13. The method of claim 7 wherein the exogenous ganglioside is administered in a dosage of at least 30 μg/mL.

14. The method of claim 7 wherein the administration of exogenous ganglioside comprises the step of contacting the prostate cancer cells with the exogenous ganglioside.

15. The method of claim 7 wherein the exogenous ganglioside comprises GD3-enriched zeta dairy lipid powder which is administered to the individual as a dietary supplement in a dosage of at least 30 μg/mL, the inhibition of the propagation of prostate cancer cells is accomplished by apoptosis, the prostate cancer cells comprising PC-3 cells, the propagation of normal prostate cells is not inhibited, and the normal prostate cells comprising RWPE-1 cells.

16. A kit to inhibit the propagation of prostate cancer cells, the kit comprising a dietary supplement comprising exogenous ganglioside and instructions for use of the dietary supplement.

17. The kit of claim 16 wherein the exogenous ganglioside comprises GD3.

18. The use of claim 17 wherein the GD3 comprises GD3-enriched zeta dairy lipid powder.

19. The kit of claim 16 wherein the prostate cancer cells are PC-3 cells.

20. The kit of claim 16 wherein propagation of normal prostate cells is not inhibited.

21. The kit of claim 16 wherein the exogenous ganglioside is administered to an individual in need of such treatment in a dosage of at least 30 μg/mL.
